(19) United States
(12) Patent Application Publication
(10) Pub. No.: US 2020/0268889 A1
(43) Pub. Date: Aug. 27, 2020

(54) (R)-12-HYDROXYSTEARIC ACID HYDRAZIDES AS GELATORS AND SELF-STANDING GELS THEREOF

(71) Applicant: Georgetown University, Washington, DC (US)

(72) Inventors: Jingjing LI, Washington, DC (US); Richard G. Weiss, Bethesda, MD (US)

(73) Assignee: Georgetown University, Washington, DC (US)

(21) Appl. No.: 15/781,053

(22) PCT Filed: Dec. 28, 2016
(86) PCT No.: PCT/US2016/068862

§ 371 Date: Jun. 1, 2018

Related U.S. Application Data
(60) Provisional application No. 62/271,746, filed on Dec. 28, 2015.

(51) Int. Cl. A61K 47/16; C07C 243/28; A61K 45/06; A61K 9/06

(52) U.S. Cl. CPC A61K 47/16; A61K 9/06; A61K 45/06; C07C 243/28

(57) ABSTRACT

(R)-12-hydroxystearic acid hydrazides as gelators and gels generated therefrom are provided. A series of (R)-12-hydroxystearic acid hydrazides produce gels that are self-standing and self-healing. The (R)-12-hydroxystearic acid hydrazides are capable of exhibiting gelling properties in a wide variety of solvents. The gels have demonstrated good potential for use in drug release.

### CROSS-REFERENCE TO RELATED APPLICATION

This application is related to and claims the benefit of U.S. Provisional Application No. 62/271,746, entitled “(R)-12-HYDROXYSTEARIC ACID HYDRAZIDES AS VERY EFFICIENT GELATORS AND THEIR SELF-STANDING GELS” filed 28 Dec. 2015, the contents of which are incorporated herein by reference in their entirety for all purposes.

### FIELD OF THE INVENTION

The invention relates generally to (R)-12-hydroxystearic acid hydrazides as gelators and the use thereof to generate self-standing molecular gels.

### BACKGROUND OF THE INVENTION

Molecular gels typically consist of a low molecular weight gelator (LMWG) and a liquid. To date, relatively few LMWGs have been found which have crystalline, self-assembled fibrillar networks (SAFINs) and show a high degree of thixotropy (i.e., they are able to substantially reform a large part of their viscoelasticity after the cessation of destructive strain).

Of particular interest also are super-gelators, LMWGs capable of gelating liquids at concentrations below 1.0 wt %, and those forming free-standing, shape-persistent gels. Apart from polymer gels (most of which are hydrogels), known LMWGs capable of forming self-standing molecular gels are normally metal complexes with strong metal-ligand interactions, derivatives of crown ethers that undergo host-guest interactions, or amides, peptides and sugars with strong hydrogen-bonding units. Such super-gelators also include highly luminescent oxadiazole-based stilbene molecules without hydrogen-bonding motifs. The SAFINs of these self-standing molecular gels in low-polarity liquids derive principally from intermolecular Π-Π interactions. Designing gelators with these desirable properties remains a huge challenge, and most have been found serendipitously.

A problem with many of these gelators, the solvents in which they produce gels, and their resulting gels themselves is that they lack biocompatibility; that is, they are unsuitable for contact or use with living tissue, such as, for example, contact with human skin or mucosa. There exists a need to develop highly efficient gelators capable of producing molecular gels, including self-standing gels, in, for example, the medical and pharmaceutical fields for potential drug delivery applications. A key challenge in these fields that must be addressed is that the gelators need to be capable of causing gelation in biocompatible solvents such that resulting molecular gels are safe for medical use.

### SUMMARY OF THE INVENTION

The invention provides (R)-12-hydroxystearic acid hydrazides and gels derived therefrom. The (R)-12-hydroxystearic acid hydrazides are versatile and can produce gels in various solvents.

In some embodiments, a series of (R)-12-hydroxystearic acid hydrazides produce gels that are self-standing, self-healing, thixotropic, load-bearing and have moldable properties.

The gels may have self-assembled fibrillar networks (SAFINs) that are crystalline in structure. In some embodiments, the gels have demonstrated good potential for use in drug release.

### BRIEF DESCRIPTION OF THE DRAWINGS

FIG. 1 is an illustration of the structure of (R)-12-hydroxystearic acid hydrazides in accordance with aspects of the present invention.

FIG. 2 is a schematic illustration of the synthesis of (R)-12-hydroxystearic acid hydrazide (0-HSAH) in accordance with aspects of the present invention.

FIG. 3 is a schematic illustration of the synthesis of (R)—N′-ethyl-12-hydroxyoctadecane hydrazide (2-HSAH) in accordance with aspects of the present invention.

FIG. 4 is a schematic illustration of the synthesis of (R)—N′-hexyl-12-hydroxyoctadecane hydrazide (6-HSAH) in accordance with aspects of the present invention.

FIG. 5 is a schematic illustration of the synthesis of (R)—N′-decyl-12-hydroxyoctadecane hydrazide (10-HSAH) in accordance with aspects of the present invention.

FIG. 6A is a photographic depiction of a self-standing gel block of 5 wt % 0-HSAH in ethylene glycol, in accordance with aspects of the present invention.

FIG. 6B is a photographic depiction of a self-standing gel block of 2 wt % 0-HSAH in ethylene glycol, in accordance with aspects of the present invention.

FIG. 6C is a photographic depiction of two self-standing gel blocks of 5 wt % 0-HSAH in ethylene glycol placed in contact with each other, with the lower block containing methylene blue, and the two self-standing gel blocks after 17 hours, in accordance with aspects of the present invention.

FIG. 7 is a photographic depiction of a self-standing 2 wt % 0-HSAH in propylene glycol gel, in accordance with aspects of the present invention.

FIG. 8 is a photographic depiction of a self-standing 5 wt % 0-HSAH in propylene glycol gel, in accordance with aspects of the present invention.

FIG. 9 is a photographic depiction of a self-standing 5 wt % 0-HSAH in glycerol gel, in accordance with aspects of the present invention.

FIG. 10 is a photographic depiction of a visual test for thixotropy of a 2 wt % O-HSAH in ethylene glycol gel, in accordance with aspects of the present invention.

FIG. 11A is a graphical depiction of a thixotropic study of a fast-cooled 5 wt % 0-HSAH in silicone oil gel sample, in accordance with aspects of the present invention.

FIG. 11B is a graphical depiction of a thixotropic study of a fast-cooled 2 wt % 0-HSAH in ethylene glycol gel sample, in accordance with aspects of the present invention.

FIG. 11C is a graphical depiction of a thixotropic study of a fast-cooled 5 wt % 2-HSAH in silicone oil gel sample, in accordance with aspects of the present invention.

FIG. 11D is a graphical depiction of a thixotropic study of a fast-cooled 5 wt % 6-HSAH in silicone oil gel sample, in accordance with aspects of the present invention.

FIG. 11E is a graphical depiction of a thixotropic study of a fast-cooled 5 wt % 10-HSAH in silicone oil gel sample, in accordance with aspects of the present invention.

FIG. 11F is a graphical depiction of a thixotropic study of a second fast-cooled 5 wt % 0-HSAH in silicone oil gel sample, in accordance with aspects of the present invention.

FIG. 12A is a graphical illustration of log-log plots for angular frequency sweeps at 0.05% strain in silicone oil gels at 25° C. for 0-HSAH, 2-HSAH, 6-HSAH, and 10-HSAH, in accordance with aspects of the present invention.

FIG. 12B is a graphical illustration of log-log plots for strain sweeps at 1 Hz frequency in silicone oil gels at 25° C. for 0-HSAH, 2-HSAH, 6-HSAH, and 10-HSAH, in accordance with aspects of the present invention.

FIG. 13 is a graphical illustration of the recovery of G′ (average of five consecutive thixotropic recover measurements) of 5 wt % 0-HSAH in silicone oil gel at 25° C. and best single exponential decay fit line, in accordance with aspects of the present invention.

FIG. 14A is a graphical illustration of log-log plots for angular frequency sweeps at 0.05% strain for 0-HSAH in ethylene glycol gels at 25° C., in accordance with aspects of the present invention.

FIG. 14B is a graphical illustration of log-log plots for strain sweeps at 1 Hz frequency for 0-HSAH in ethylene glycol gels at 25° C., in accordance with aspects of the present invention.

FIG. 15A is a graphical illustration of a log-log plot of the viscosity versus shear rate for 5 wt % 0-HSAH in ethylene glycol gels at 25° C., in accordance with aspects of the present invention.

FIG. 15B is a graphical illustration of a log-log plot of the shear stress versus shear rate for 5 wt % 0-HSAH in ethylene glycol gels at 25° C., in accordance with aspects of the present invention.

FIG. 16A is a graphical depiction of compression experiment curves of normal force versus gap distance for gels of 2 wt % and 5 wt % 0-HSAH in ethylene glycol (EG) or EG-DMF mixtures, in accordance with aspects of the present invention.

FIG. 16B is a graphical depiction of compression and extension curves of 5 wt % 0-HSAH in ethylene glycol gels (black) and repeated cycles of compression (squares) and extension (circles), below Fb, in accordance with aspects of the present invention.

FIG. 17A is a graphical illustration of compression (squares) and extension (circles) curves for 2 wt % 0-HSAH in ethylene glycol, in accordance with aspects of the present invention.

FIG. 17B is a graphical illustration of compression (squares) and extension (circles) curves for 5 wt % 0-HSAH in 8:2 (v/v) ethylene glycol:DMF mixtures, in accordance with aspects of the present invention.

FIG. 18A is a polarized optical microscopy (POM) image of a slow-cooled 5 wt % 0-HSAH in ethylene glycol gel, in accordance with aspects of the present invention.

FIG. 18B is a polarized optical microscopy (POM) image of a fast-cooled 5 wt % 0-HSAH in ethylene glycol gel, in accordance with aspects of the present invention.

FIG. 18C is a polarized optical microscopy (POM) image of a slow-cooled 5 wt % 0-HSAH in silicone oil gel, in accordance with aspects of the present invention.

FIG. 18D is a polarized optical microscopy (POM) image of a fast-cooled 5 wt % 0-HSAH in silicone oil gel, in accordance with aspects of the present invention.

FIG. 18E is a polarized optical microscopy (POM) image of a slow-cooled 5 wt % 2-HSAH in silicone oil gel, in accordance with aspects of the present invention.

FIG. 18F is a polarized optical microscopy (POM) image of a fast-cooled 5 wt % 2-HSAH in silicone oil gel, in accordance with aspects of the present invention.

FIG. 18G is a polarized optical microscopy (POM) image of a slow-cooled 5 wt % 6-HSAH in silicone oil gel, in accordance with aspects of the present invention.

FIG. 18H is a polarized optical microscopy (POM) image of a fast-cooled 5 wt % 6-HSAH in silicone oil gel, in accordance with aspects of the present invention.

FIG. 18I is a polarized optical microscopy (POM) image of a slow-cooled 5 wt % 10-HSAH in silicone oil gel, in accordance with aspects of the present invention.

FIG. 18J is a polarized optical microscopy (POM) image of a fast-cooled 5 wt % 10-HSAH in silicone oil gel, in accordance with aspects of the present invention.

FIG. 19A is an XRD diffractogram of 0-HSAH powder, 10 wt % 0-HSAH in silicone oil gel, and 5 wt % 0-HSAH in ethylene glycol gel, in accordance with aspects of the present invention.

FIG. 19B is an XRD diffractogram of 2-HSAH powder and 10 wt % 2-HSAH in silicone oil gel in accordance with aspects of the present invention.

FIG. 19C is an XRD diffractogram of 6-HSAH powder and 10 wt % 6-HSAH in silicone oil gel in accordance with aspects of the present invention.

FIG. 19D is an XRD diffractogram of 10-HSAH powder and 10 wt % 10-HSAH in silicone oil gel in accordance with aspects of the present invention.

FIG. 20A is a depiction of FT-IR spectra of neat silicone oil, neat 0-HSAH powder, 5 wt % 0-HSAH in silicone oil gel, and 2 wt % 0-HSAH in CHCl₃ solutions/sols, in accordance with aspects of the present invention.

FIG. 20B is a depiction of FT-IR spectra of neat silicone oil, neat 2-HSAH powder, 5 wt % 2-HSAH in silicone oil gel, and 2 wt % 2-HSAH in CHCl₃ solutions/sols, in accordance with aspects of the present invention.

FIG. 20C is a depiction of FT-IR spectra of neat silicone oil, neat 6-HSAH powder, 5 wt % 6-HSAH in silicone oil gel, and 2 wt % 6-HSAH in CHCl₃ solutions/sols, in accordance with aspects of the present invention.

FIG. 20D is a depiction of FT-IR spectra of neat silicone oil, neat 10-HSAH powder, 5 wt % 10-HSAH in silicone oil gel, and 2 wt % 10-HSAH in CHCl₃ solutions/sols, in accordance with aspects of the present invention.

FIG. 21A is an illustration of a possible molecular packing model for 0-HSAH, in accordance with aspects of the present invention.

FIG. 21B is an illustration of a possible molecular packing model for 10-HSAH, in accordance with aspects of the present invention.

FIG. 22 is a depiction of FT-IR spectra of (R)-HSA, O-HSAH, (R)-12-hydroxyoctadecanamide (0-HSAA), (R)-12-hydroxy-N-propyloctadecanamide (3-HSAA), (R)-12-hydroxy-N-octadecyloctadecanamide (18-HSAA), 10-HSAH, and (R)-18-(pentylamino)octadecan-7-ol (HSN-5) powders, in accordance with aspects of the present invention.

FIG. 23 is a depiction of XRD diffractograms of 0-HSAH, (R)-HSA, 10-HSAH, and HSN-5 powders, in accordance with aspects of the present invention.

FIG. 24 is a photographic depiction of the experimental setup for measurement of the diffusion coefficient of (a) methylene blue and (b) erythrosine B.

FIG. 25A is a graphical illustration of absorption spectra of methylene blue in ethylene glycol.

FIG. 25B is a graphical illustration of a UV-vis calibration curve of methylene blue in ethylene glycol.

FIG. 25C is a graphical illustration of absorption spectra of erythrosine B in ethylene glycol.

FIG. 25D is a graphical illustration of a UV-vis calibration curve of erythrosine B in ethylene glycol.

FIG. 26A is a graphical illustration of absorption spectra of methylene blue released from 2 wt % 0-HSAH/ethylene glycol gel blocks at different times at 25° C., in accordance with aspects of the present invention.

FIG. 26B is a graphical illustration of absorption spectra of erythrosine B released from 2 wt % 0-HSAH/ethylene glycol gel blocks at different times at 25° C., in accordance with aspects of the present invention.

FIG. 27A is a photographic and graphical illustration of a diffusion plot of Mₜ² versus t for methylene blue diffusion from a 2 wt % 0-HSAH in ethylene glycol gel into ethylene glycol liquid at 25° C., in accordance with aspects of the present invention.

FIG. 27B is a photographic and graphical illustration of a diffusion plot of Mₜ² versus t for erythrosine B diffusion from a 2 wt % 0-HSAH in ethylene glycol gel into ethylene glycol liquid at 25° C., in accordance with aspects of the present invention.

### DETAILED DESCRIPTION OF THE INVENTION

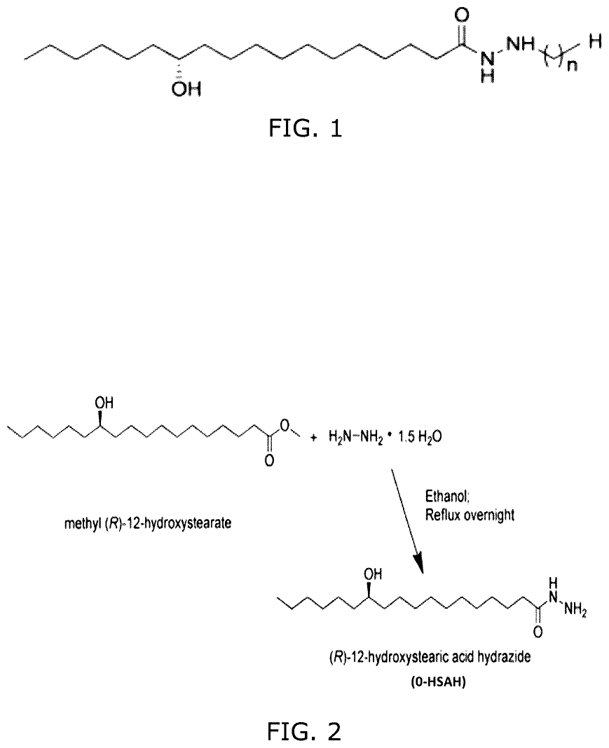

The present invention generally relates to a series of hydrazides (“n-HSAH”) derived from (R)-12-hydroxystearic acid (FIG. 1), and to gels derived therefrom. The structure of the n-HSAH compounds is shown below, wherein n is any integer from 0 to 20 inclusive.

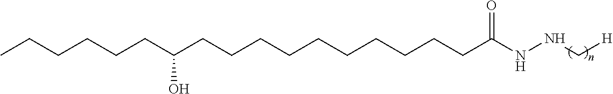

The integer n may be zero, or it may be an odd or even number. For each gel employing an n-HSAH according to the invention, the (S) isomer of the n-HSAH may also be present in an amount less than or equal to the amount of (R) isomer. Therefore, the (R) isomer constitutes at least 50% of the total of (R) and (S) isomers of any given hydrazide present in the gel. It may constitute at least 60%, or at least 70%, 80%, 90%, 95%, 98%, or 99% of the total. Gelators other than n-HSAH compounds may optionally be included in the gel, or they may be excluded.

Non-limiting examples of liquids that may be gelled with n-HSAH compounds according to the invention include water, organic liquids, and combinations thereof, for example solutions of organic liquids in water or vice versa. Exemplary organic liquids include C1-C10 aliphatic hydrocarbons (for example hexane and decane), silicone oils, toluene, each of the xylene isomers, CHCl₃, chlorobenzene, ethyl acetate, THF, 1-butanol, ethanol, methanol, 1-propanol, 2-propanol, 1-butanol, 2-butanol, isobutyl alcohol, 1-octanol, nitrobenzene, DMF, acetonitrile, ethylene glycol, propylene glycol, glycerol, DMSO, propylene carbonate, and combinations thereof.

The n-HSAH compounds disclosed herein may form gels in a variety of liquids by self-assembly mechanisms that appear to depend on the specific value of n and the choice of liquid. This comportment is similar in some respects to those of the analogous (R)—N-alkyl-12-hydroxystearamides, but the specific properties of the hydrazides are very different in some important respects. Thus, in several of the hydrazides, the properties of the gels can be correlated with their mechanisms of formation. Notably, the parent hydrazide, (R)-12-hydroxystearic acid hydrazide (0-HSAH), was found to be a super-gelator in ethylene glycol, propylene glycol, and glycerol, and its gels exhibited self-standing, self-healing, moldable and load-bearing properties that are not found in the corresponding amide. Although other molecular gels have been shown to possess one or more of the properties reported here, none possesses all of them in one gel with such simply-structured gelators.

### EXAMPLES

### Example 1. Synthesis of (R)-12-Hydroxystearic Acid Hydrazides

Certain derivatives of (R)-12-hydroxystearic acid hydrazides were specifically studied, including n-HSAH derivatives in which n=0, 2, 6, or 10.

For synthesizing (R)-12-hydroxystearic acid hydrazide (0-HSAH), the parent hydrazide, a mixture of methyl (R)-12-hydroxystearate (10.0 g, 3.2 mmol) and hydrazine hydrate (10.0 g, 16.9 mmol) was heated in ethanol (90 mL). After refluxing overnight, the hot solution was poured into 500 mL water with stirring. The precipitate was washed with 3×200 mL water and dried in vacuum to give 8.9 g (89%) of a pale yellow powder. 5.0 g of this material was refluxed twice in 100 g aliquots of hexane for 30 min. The solution was filtered hot. The filter cake was washed with a small amount of hexane and then recrystallized from ethyl acetate (200 g) and methanol (100 g) mixtures twice to give 2.2 g (44%) 0-HSAH as a white powder. FIG. 2.

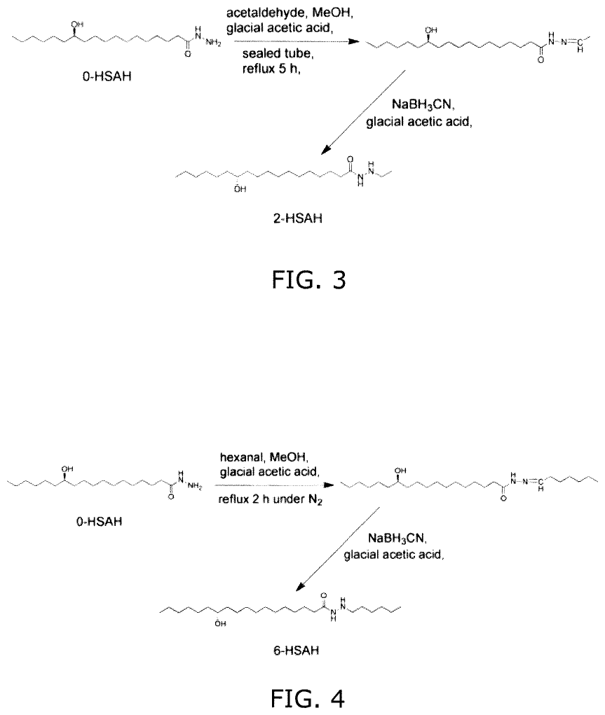

For synthesizing (R)—N′-ethyl-12-hydroxyoctadecane hydrazide (2-HSAH), 0-HSAH (2.0 g, 6.4 mmol) and acetaldehyde (1.7 g, 38.6 mmol) were mixed in methanol (7.0 g) in a thick glass tube at 0° C. Glacial acetic acid (0.8 g) was added as a catalyst. The tube was sealed with a screw cap and the mixture was stirred for 5 hours at 70° C. After cooling, the product was filtered and recrystallized from 30 g methanol to give 1.3 g (60%) of two geometric isomers of (R)—N′-ethylidene-12-hydroxy octadecane hydrazide. (R)—N′-ethylidene-12-hydroxy octadecane hydrazide (0.43 g, 1.26 mmol) was dissolved in 5 mL methanol at room temperature and stirred for 30 min. Sodium cyanoborohydride (156 mg, 2.48 mmol) and glacial acetic acid (156 mg, 2.60 mmol) were dissolved in 1 mL methanol and added drop-wise to the methanol solution over 10 minutes under a N₂ atmosphere and then stirred for 5 hours at 0° C. and for 45 minutes at room temperature. Thereafter, the mixture was filtered. The filter cake was washed with water and recrystallized from ethyl acetate. The crude product was extracted with CHCl₃, dried under vacuum and then recrystallized from a mixture of ethyl acetate (200 g) and methanol (100 g) twice to give 60 mg (14%) 2-HSAH. FIG. 3.

For synthesizing (R)—N′-hexyl-12-hydroxyoctadecane hydrazide (6-HSAH), a mixture of 0-HSAH (3.1 g, 9.8 mmol) and hexanal (1.5 g, 15.0 mmol) was heated in methanol (30 mL). Glacial acetic acid (1.0 g) was added as a catalyst to the system. The mixture was refluxed under N₂ atmosphere for 2 hours. After cooling, the product was filtered and recrystallized from methanol to give 2.3 g (60%) of two geometric isomers of (R)—N′-hexylidene-12-hydroxyoctadecanehydrazide. (R)—N′-Hexylidene-12-hydroxyoctadecane hydrazide (2.30 g, 5.80 mmol) was dissolved in 50 mL methanol at room temperature and stirred for 30 min. Sodium cyanoborohydride (0.74 g, 11.78 mmol) and glacial acetic acid (0.74 g, 12.32 mmol) were dissolved in 20 mL methanol and added drop-wise to the methanol solution over 30 minutes under a N₂ atmosphere and then stirred for 1 hour at 0° C. and for 2 hours at room temperature. Thereafter, the mixture was filtered. The filter cake was washed with methanol and recrystallized from methanol. The crude product was extracted with CHCl₃, dried under vacuum and then recrystallized from methanol to give 480 mg (20%) 6-HSAH. FIG. 4.

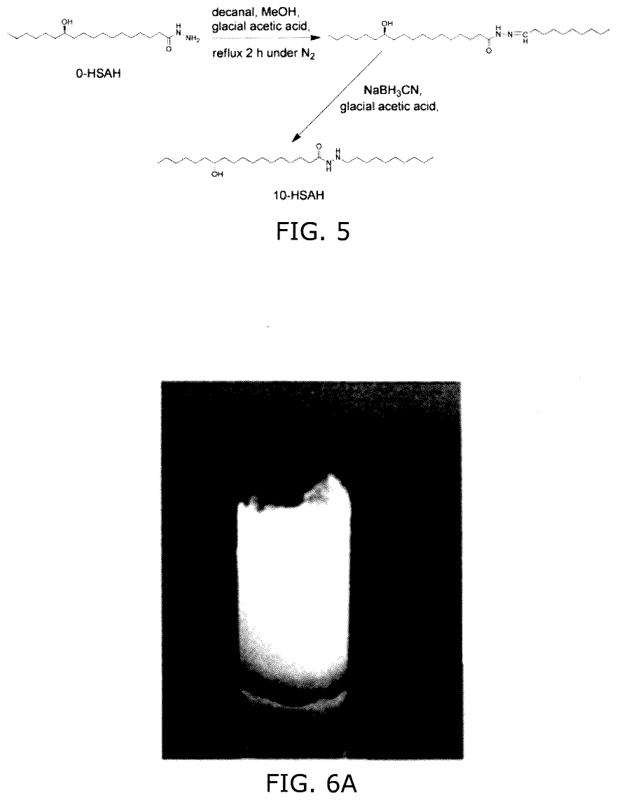

For synthesizing (R)—N′-decyl-12-hydroxyoctadecane hydrazide (10-HSAH), a mixture of 0-HSAH (1.0 g, 3.2 mmol) and decanal (0.7 g, 4.5 mmol) was heated in methanol (10 mL). Glacial acetic acid (0.3 g) was added as a catalyst to the system. The mixture was refluxed under N₂ atmosphere for 2 hours. After cooling, the product was filtered and recrystallized from methanol to give 1.2 g (83%) of two geometric isomers of (R)—N′-decylidene-I2-hydroxyoctadecane hydrazide. (R)—N′-Decylidene-12-hydroxyoctadecane hydrazide (0.20 g, 0.44 mmol) was dissolved in 20 mL methanol at room temperature and stirred for 30 minutes. Sodium cyanoborohydride (0.06 g, 0.95 mmol) and glacial acetic acid (0.06 g, 0.10 mmol) were dissolved in 10 mL methanol and added drop-wise into the methanol solution over 30 minutes under a N₂ atmosphere and then stirred for 5 hours at room temperature. Thereafter, the mixture was filtered. The filter cake was washed with methanol and recrystallized from methanol. The crude product was extracted with CHCl₃, dried under vacuum and then recrystallized from methanol to give 0.10 g (50%) 10-HSAH. FIG. 5.

### Example 2. Preparations of Gels from (R)-12-Hydroxystearic Acid Hydrazides

Known amounts of an (R)-12-hydroxystearic acid hydrazide gelator and a liquid were heated in a flame-sealed 5 mm (inside diameter) tube to 120° C. (i.e., slightly higher than the melting points of the gelators). Then, the hot tube was taken from the bath and left undisturbed at room temperature overnight. Samples that formed two layers at 120° C. are labeled “insoluble” (I); samples that dissolved after heating, but precipitated or formed viscous liquids when cooled, are classified as “precipitates” (P) or “viscous” (V), respectively. Those that appeared to be clear or only microscopically heterogeneous and did not flow when inverted are considered (by preliminary classification) to be gels (G). The critical gelator concentrations (CGCs) were determined by adding sequentially a small amount of liquid into the glass tubes that appeared to contain gels. After each addition, the tubes were resealed, reheated and recooled as described above. The CGCs were the lowest concentrations at which the inverted samples did not flow when inverted.

Gel-to-sol transition temperatures (T_{geis}) at 5 wt % gelator were also determined by the invert tube method. The heating rate for the T_{geis} test was 2° C./min in an oil bath. The temperature range was recorded from when the first drop of liquid fell to when the entire gel collapsed. For preparation of the self-standing gel blocks, hot solutions/sols of 2 wt % or 5 wt % 0-HSAH in ethylene glycol were poured into a two channel syringe; the gels were formed within 5 minutes. After 30 minutes, the gel was pushed out with the syringe plunger and the gel block was cut into two pieces. One was submerged into a 500 mg/L methylene blue-ethylene glycol solution for 10 min. The excessive solution on the surface of the gel block was absorbed with a tissue. Then, the two pieces were put together again and allowed to stand overnight to assess the self-healing properties. If they remained one piece when suspended horizontally, they were considered joined.

The gelation behaviors of 5 wt % n-HSAH (n=0, 2, 6, 10) in different liquids are summarized in Table 1. 0-HSAH shows much better gelation properties than the other gelators of the series. It can gelate a wide variety of liquids, including alkanes, alcohols, aromatic liquids, and even DMSO. By contrast, the other n-HSAH were unable to form gels in alkane liquids like hexane and decane and most of the alcohol liquids (except ethylene glycol). However, they were able to gelate aromatic liquids, DMSO and propylene carbonate. In addition, the CGCs of the gels formed by 0-HSAH are generally lower than those of the other n-HSAH, especially in ethylene glycol, in which the CGC of 0-HSAH was astonishingly low, 0.06 wt %. By contrast and demonstrating the importance of the hydroxyl group at C12, stearic acid hydrazide (SAH), the gelator analogous to 0-HSAH, but lacking a hydroxyl group at C12, required a minimum of 3 wt % to gelate ethylene glycol.

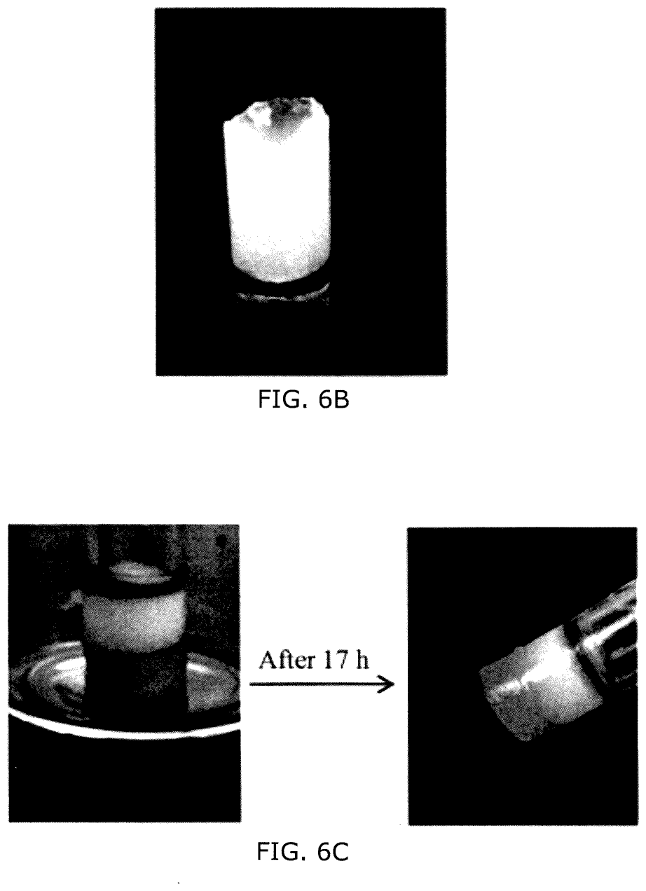

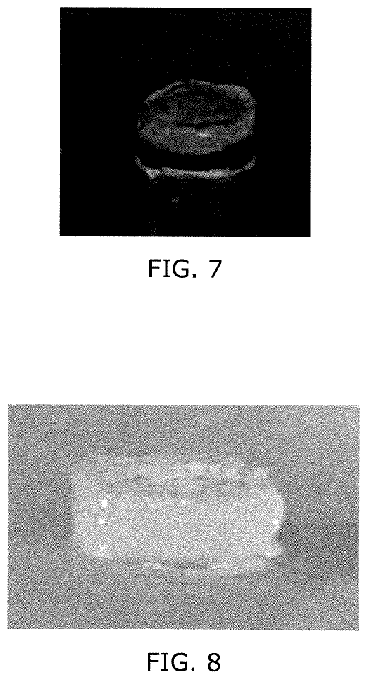

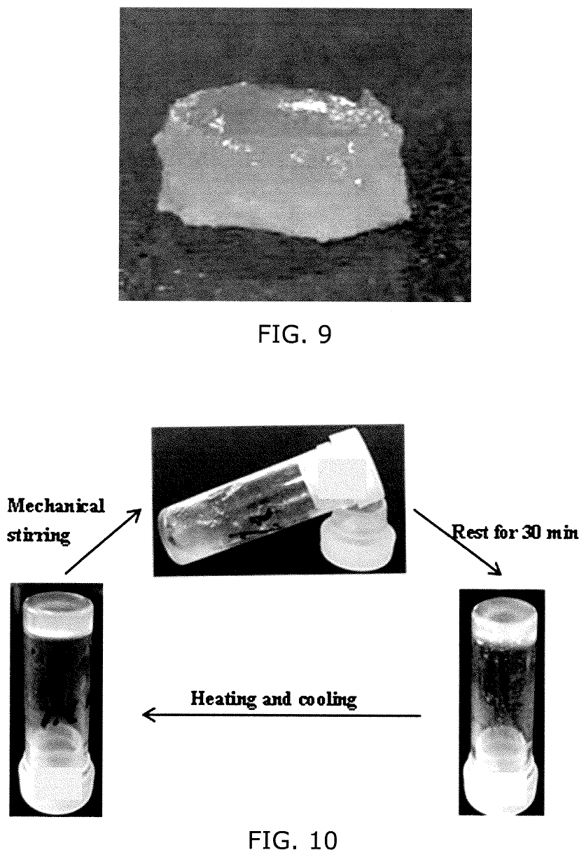

The 0-HSAH in ethylene glycol gels also showed self-standing, self-healing, thixotropic, load-bearing and moldable properties (FIGS. 6A, 6B, and 10). FIG. 10 demonstrates a visual test for thixotropy in a 2 wt % 0-HSAH in ethylene glycol gel. The 2 wt % 0-HSAH in ethylene glycol gel, made by the heating-cooling method discussed above (heating in a flame-sealed 5 mm tube to 120° C. and removing to room temperature overnight) was agitated vigorously for several minutes with a glass rod. Thereafter, it was allowed to remain at rest for 30 minutes and the gel properties returned (i.e., a lack of flow when inverted). A self-standing cylindrical gel block was also made with 2 wt % and 5 wt % 0-HSAH in both propylene glycol and glycerol. See FIGS. 7-9.

A self-standing cylindrical gel block was made with 2 or 5 wt % 0-HSAH in ethylene glycol, but could not be made with 0.5 or 1 wt % concentrations of the gelator. To demonstrate the self-healing properties more clearly, one 5 wt % 0-HSAH in ethylene glycol gel block was cut into two pieces and one of the pieces was submerged into a solution of methylene blue in ethylene glycol for 10 min. Thereafter, the two pieces were placed in contact as shown in FIG. 6C. After 17 hours, the two pieces had become one. A blue color in the upper gel piece indicated some diffusion of methylene blue (and ethylene glycol) between the two pieces.

All of the n-HSAH derivatives were able to gelate silicone oil. As such and because it has a high boiling point and low volatility, silicone oil was selected as the liquid to study further the gelation properties of the series of hydrazides.

TABLE 1
Appearances^{a} of 5 wt % n-HSAH in different liquids and the CGCs
(wt %) and T_{gels} (° C.) values of their fast-cooled gels.

| Liquid | 0-HSAH | 2-HSAH | 6-HSAH | 10-HSAH |
|---|---|---|---|---|
| hexane | OG (3.9%, | P | P | P |
| | 107-108) | | | |
| decane | OG (1.6%, | P | P | P |
| | 109-111) | | | |
| CHCl₃ | OG (1.1%, | OG | P | P |
| | 80-83) | | | |
| ethyl acetate | P | P | P | P |
| acetonitrile | P | P | P | P |
| THF | P | P | P | P |
| methanol | TG^{d} | P | P | P |
| ethanol | TG^{d} | P | P | P |
| ethylene glycol | OG (0.06%, | OG (1.1%, | OG (0.8%, | I |
| | 83-85) | 93-94) | 95-98) | |
| 1-butanol | OG (3.0%, | V | V | V |
| | 38-40) | | | |
| 1-octanol | OG (2.8%, | V | V | V |
| | 53-55) | | | |
| toluene | TG (3.7%, | TG (3.1%, | TG (3.2%, | OG (3.3%, |
| | 75-78) | 72-75) | 68-71) | 68-71) |
| chlorobenzene | CG (0.6% | TG (2.4%, | TG (1.9%, | TG (2.5%, |
| | 80-81) | 65-68) | 66-68) | 63-65) |
| nitrobenzene | OG (0.9%, | OG (2.4%, | OG (0.4%, | V |
| | 78-79) | 77-78) | 75-77) | |
| DMSO | OG (1.5%. | OG (2.6%, | OG (2.3%, | OG (2.0%, |
| | 62-65) | 62-65) | 70-73) | 80-83) |
| DMF | P | P | OG (3.2%, | OG (3.7%, |
| | | | 60-63) | 68-70) |
| propylene | OG (1.5%, | OG(2.5%, | P | OG (2.5%, |
| carbonate | 90-92) | 100-101) | | 100-103) |
| silicone oil | TG (1.5%, | OG (1.9%, | OG (2.4%, | OG (3.4%, |
| | 108-109) | 99-103) | 97-101) | 98-103) |
| water | I | I | I | I |

^{a}OG = opaque gel, CG = clear gel, TG = translucent gel, P = precipitate, I = insoluble, V = viscous liquids;
^{d}gels became precipitates within 22 h;
CGCs determined to within ±0.1 wt % except the 0-HSAH in ethylene glycol gel for which the limit was ±0.01 wt %;
Data are an average of at least 2 determinations,

### Example 3. Mechanical Properties of Gels

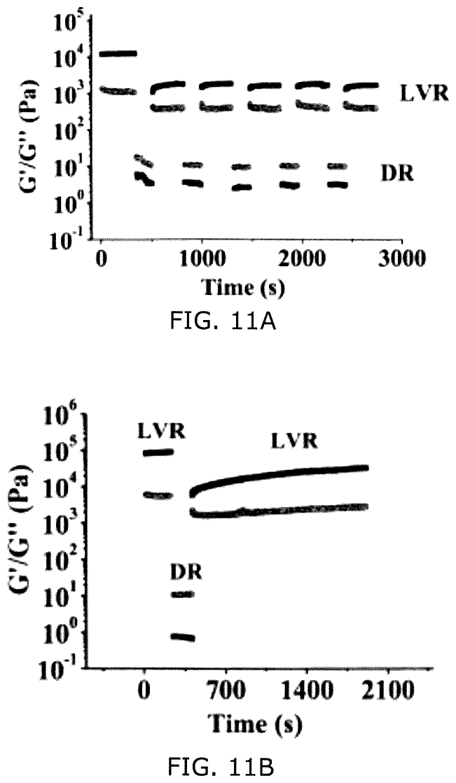

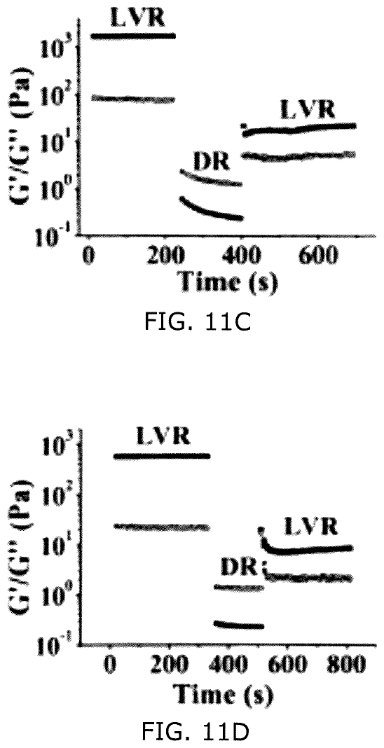

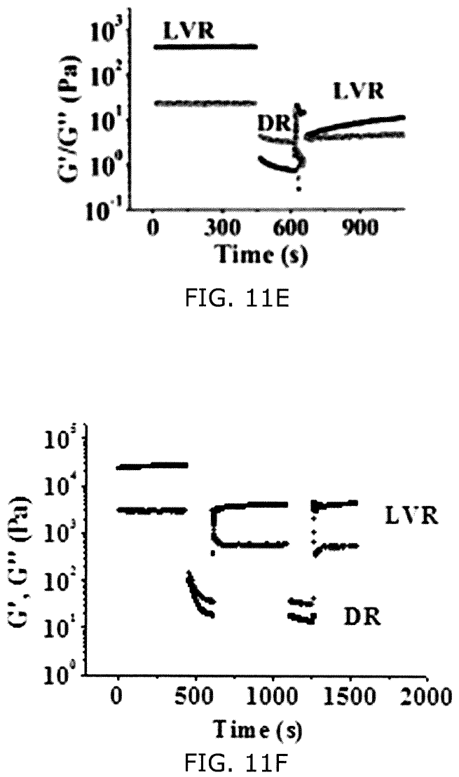

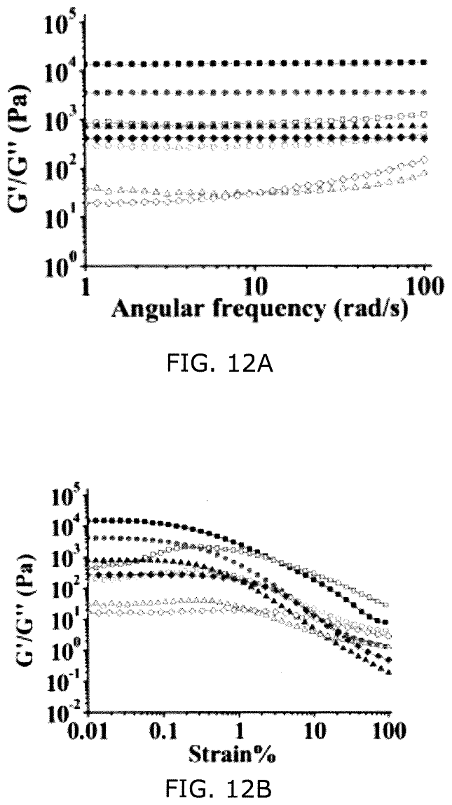

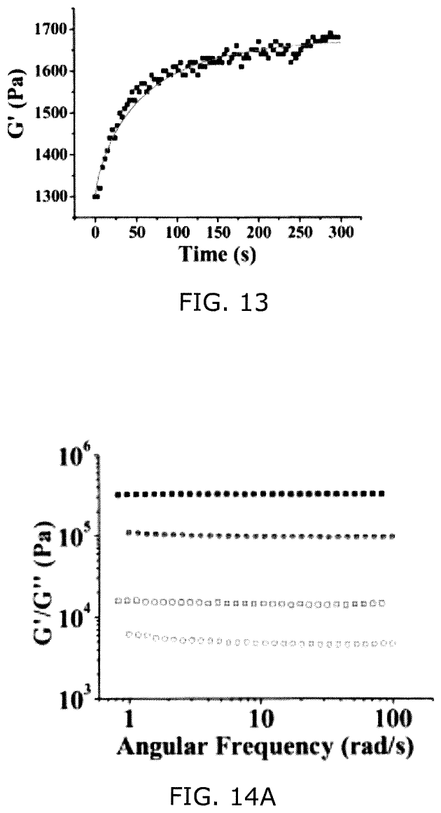

The mechanical properties of the silicone oil gels were investigated at different conditions within the linear viscoelastic regions (FIGS. 11A, 11C-11F and 12A-12B). Regardless, the values of the storage modulus (G′) of 5 wt % n-HSAH in silicone oil were always larger than the loss modulus (G″) and they remained so over very large frequency ranges; these are true gels. Consistent with the thermal properties of the gels, as reflected by the T_{gel} and CGC values, the G′ values from the 0-HSAH in silicone oil gels were much larger than those from the other n-HSAH gels; the 0-HSAH gels are much stronger. In addition, the 5 wt % 0-HSAH in silicone oil gel showed good mechanotropic recovery characteristics that were independent of the degree of destructive strain applied within the range examined: FIG. 11A for 150% destructive strain and FIG. 11F for 100% destructive strain. The actual recovery of G′ after cessation of destructive strain was 15% for the first cycle, and 95% for the second and subsequent cycles. The corresponding recovery time, based upon cycles 2-6 in FIG. 11A, was about 59 s (FIG. 13, according to, Eq 1 below, wherein m is a dimensionless constant; τ is a time constant representing the recovery speed of the partially thixotropic gels; and G′(0), G′(t) and G′(∞) are the storage moduli at time=0, an intermediate time, and time=∞, respectively, during the recovery process, i.e., after the cessation of destructive strain). However, none of the gels with 5 wt % of the other n-HSAH in silicone oil gels were thixotropic: recoveries were only about 2% and the recovered G′ values, ˜10 Pa, were too low to support the weights of the samples when they were inverted.

$$ln [ - ln \frac{{G}^{′} ( ∞ ) - {G}^{′} ( t )}{{G}^{′} ( ∞ ) - {G}^{′} ( 0 )} ] = m ln t - m ln τ \qquad ( Eq 1 )$$

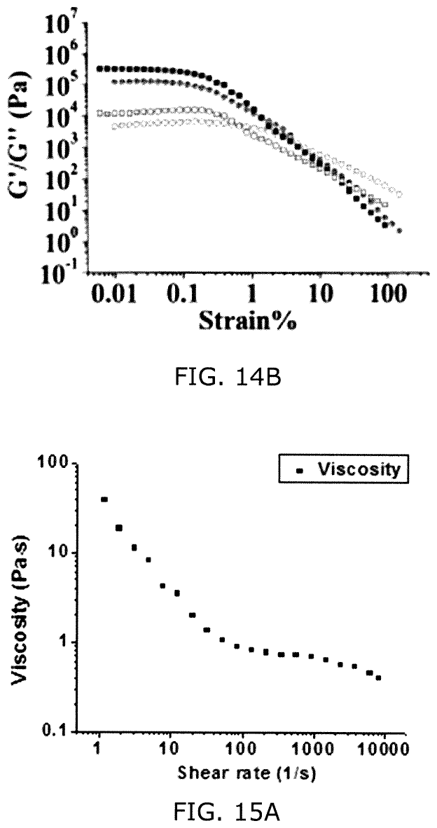

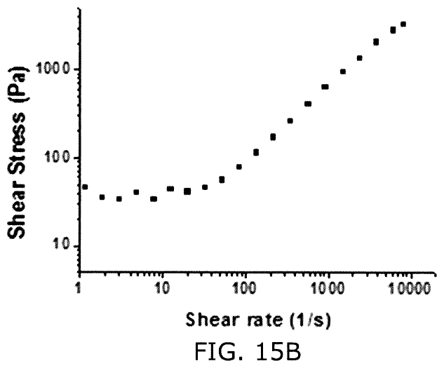

The mechanical properties of the ethylene glycol gels were also investigated under different conditions. As shown in FIGS. 14A and 14B, the storage modulus (G′) of the self-standing gels of both 2 wt % and 5 wt % 0-HSAH in ethylene glycol were >105 Pa, much stronger than that of the 5 wt % 0-HSAH gels in silicone oil (˜104 Pa), and indicating much stiffer properties for the 0-HSAH/EG gels. The strain-stress data in FIGS. 15A and 15B show shear thinning properties for the 0-HSAH/EG gels: the viscosity decreased with increasing shear rate and showed a yield stress of 35 Pa. Moreover, the 0-HSAH/EG gels also showed some thixotropic behavior (FIG. 11B): the samples recovered a part of their original viscoelastic gel properties after the cessation of destructive strain. These quantitative data are consistent with the qualitative visual tests described above. However, the recovery time was much longer than that of the 5 wt % 0-HSAH in silicone oil gels. Even 25 minutes after the cessation of destructive strain, the storage modulus (G′) had not reached a plateau value.

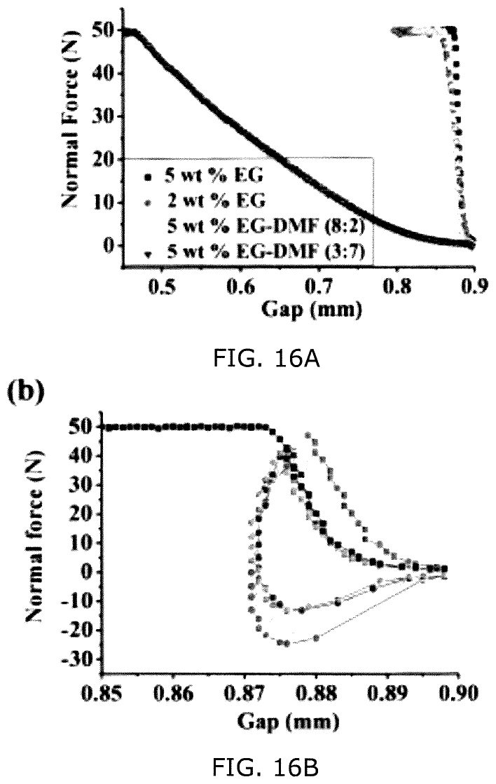

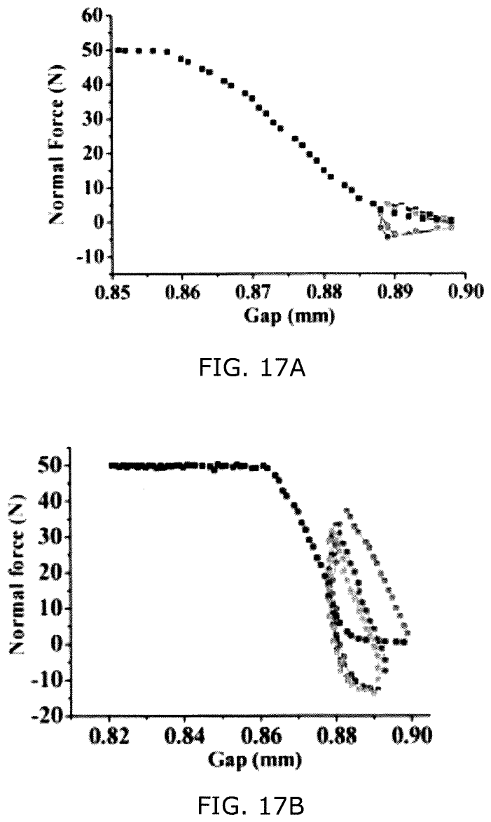

In order to test further the stiffness of the 0-HSAH in ethylene glycol gel blocks, compression rheology experiments were performed on a 0.9 mm high and 25 mm diameter cylinder gel block. As the gap between the rheometer plates decreased, the normal force increased. Also, during further compression, the ethylene glycol began to be squeezed out. The normal force at this point, the load-bearing force (Fb), represents the ability of the network to support a static load. It is the critical force below which the network is able to resist compression elastically. Above this load bearing-force, the network underwent irreversible deformation. With an additional decrease of the gap, the normal force increased further until it reached the instrument limit of 50N. As the concentration of 0-HSAH in the gel was increased from 2 wt % to 5 wt %, Fb also increased from 10N to 47N, and the rate of increase of the 5 wt % gel, 1.95 N/μm block, was also greater than that of the 2 wt % gel, 1.23 N/μm (FIGS. 16A-16B and Table 2). Below Fb, the mechanical properties of the gel blocks were reproducible, indicating some (but not complete) elasticity of the gel blocks (FIG. 16B). Although the quantitative results are expected to differ at faster or slower compression and extension speeds (as indicated by the apparent hysteresis in the data (FIG. 16B) and the fact that the systems are not completely elastic), the qualitative phenomenon would not. Self-standing gels were also made in mixtures of ethylene glycol and DMF with similar mechanical behaviors (FIGS. 16A-16B and 17A-17B). However, the stiffness of these gel blocks was weakened as the DMF content in the liquid increased.

TABLE 2
Summary of data from compression experiments.^{a}

| | ΔF/Δd | Load-bearing force |
|---|---|---|
| 0-HSAH (wt %)/liquid^{b} | (N/μm) | (F_{b}) |
| 5/EG | 1.95 | 47 N |
| 5/EG:DMF (8/2, v/v) | 1.34 | 36.7 N |
| 5/EG:DMF (3/7, v/v) | 0.12 | 0.25 N |
| 2/EG | 1.23 | 10 N |

^{a}All experiments were performed twice and the data were reproducible.
^{b}EG = ethylene glycol; DMF = N,N-dimethylformamide.

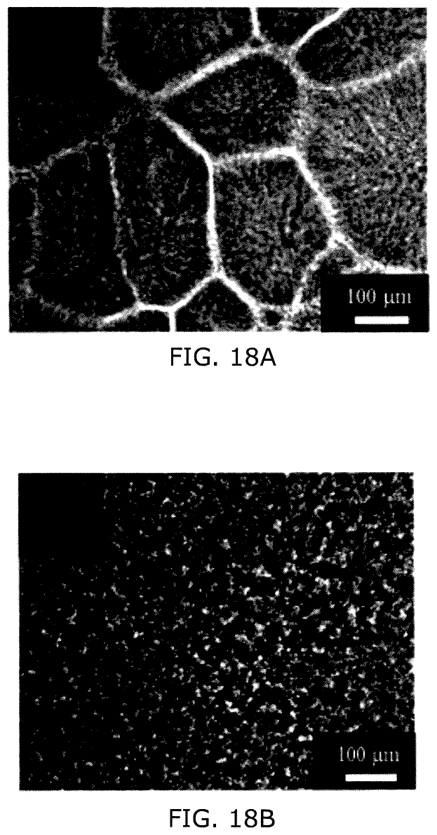

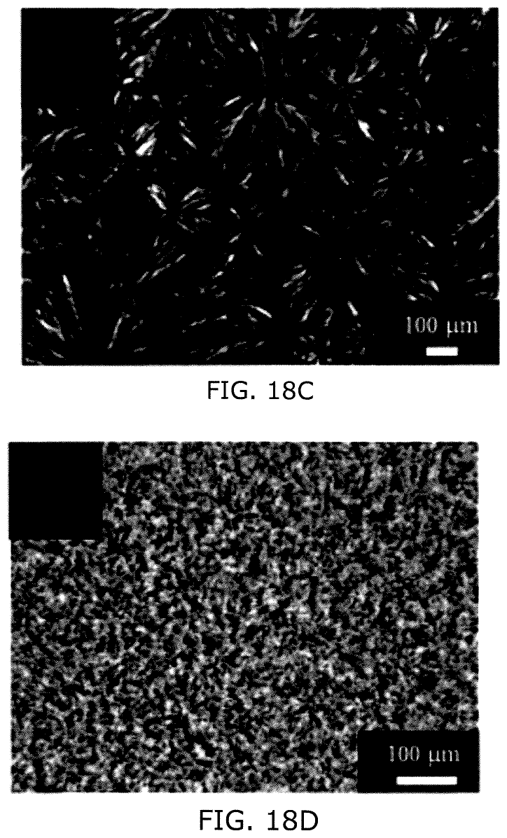

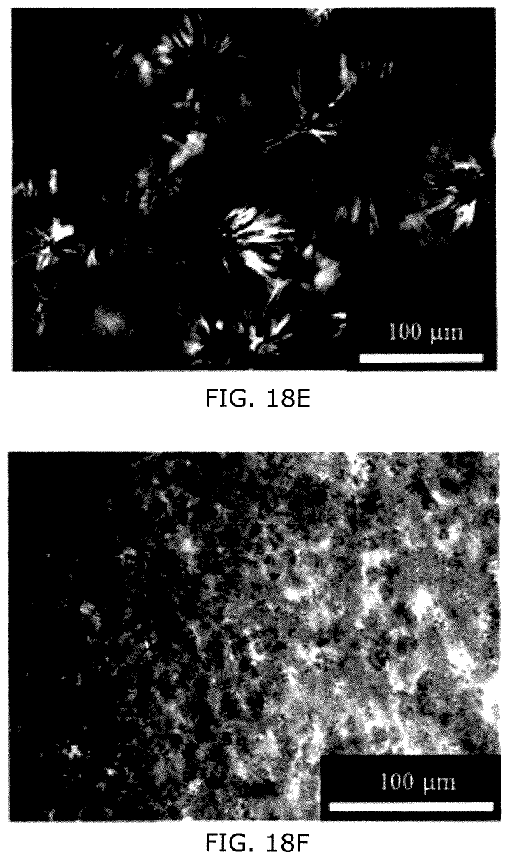

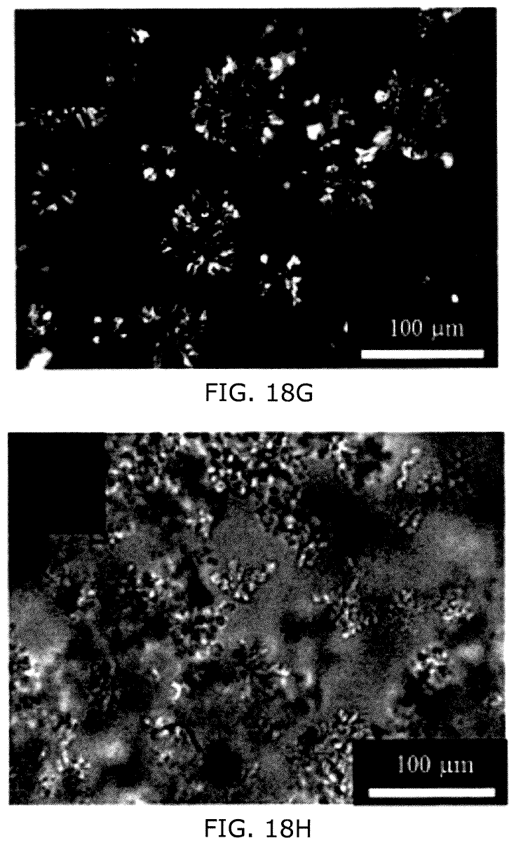

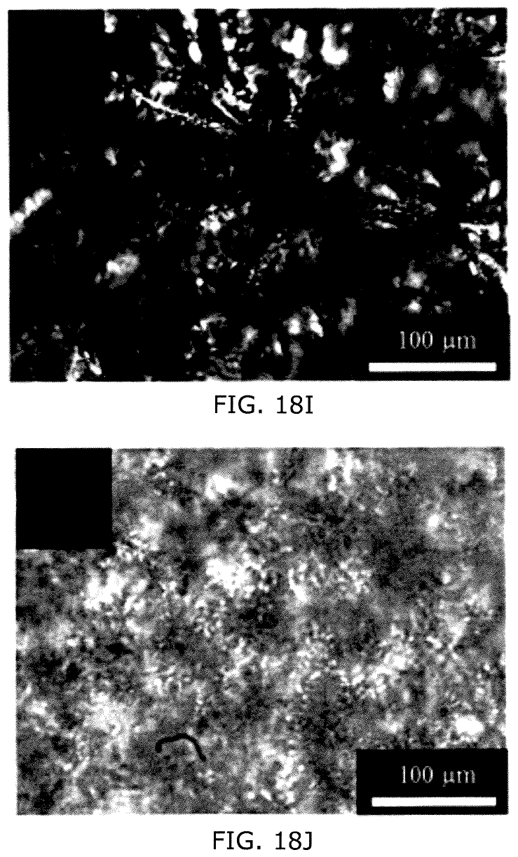

Microstructure and Molecular Packing. Polarized optical micrographs (POMs) provided information about the microstructure of the SAFINs within the gels. Hot solutions/sols were poured into 0.4 mm path-length, flattened Pyrex capillaries and flamed-sealed. The samples were reheated in an oil bath before micrographs were recorded to ensure homogeneity. For fast-cooled samples, the hot capillaries were placed in the air at room temperature overnight. For slow-cooled samples, the capillaries were kept in the oil bath at 120° C. while allowing it to decrease to room temperature slowly. The morphologies of the gels consisting of 5 wt % 0-HSAH in ethylene glycol and n-HSAH (n=0, 2, 6, 10) in silicone oil of both fast-cooled and slow-cooled samples were observed. As shown in FIGS. 18A-183, all of these gels showed spherulitic objects in which the sizes from the slow-cooled solutions/sols (FIGS. 18A, 18C, 18E, 18G, 18I) were always larger than from the fast-cooled ones (FIGS. 18B, 18D, 18F, 18H, 18J). The larger objects from slow cooling are commonly observed in SAFINs and results from slower nucleation relative to the rate of growth of objects. Although the morphologies of the 0-HSAH in silicone oil and in ethylene glycol gels for the fast-cooled samples were similar, clear differences were observed among the slow-cooled samples. The fibers of 0-HSAH in ethylene glycol gels of the slow-cooled samples were much thinner than in silicone oil gels. The very small fibers may provide stronger capillary forces and, thus, may offer an explanation for the lower CGCs of 0-HSAH in ethylene glycol than in silicone oil. As indicated in Table 1, 0-HSAH in ethylene glycol forms an opaque gel, while 0-HSAH in silicone oil forms a translucent gel. Generally, opaque gels form thicker fibers. However, the clarity of a gel is also dependent on the difference between the index of refraction between a gelator network and the liquid. If they match, a gel with even very thick fibers will appear transparent. The indices of refraction were not examined herein; only the cross-sectional dimensions of the fibers were examined.

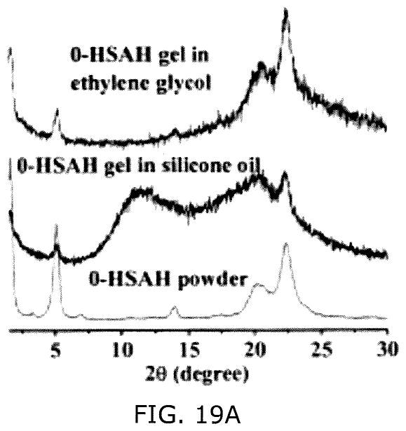

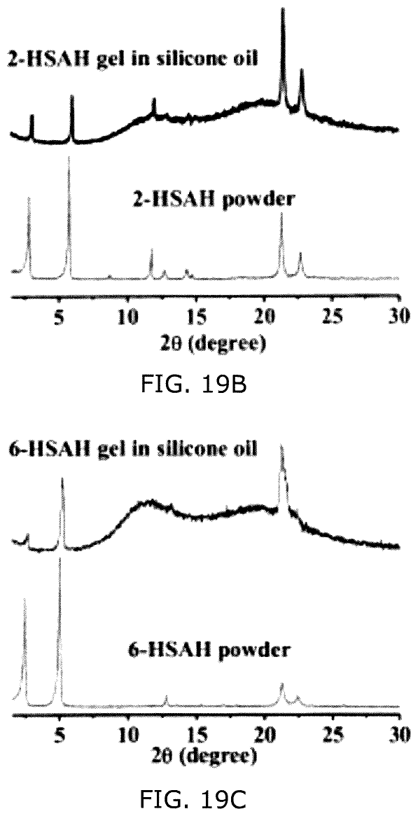

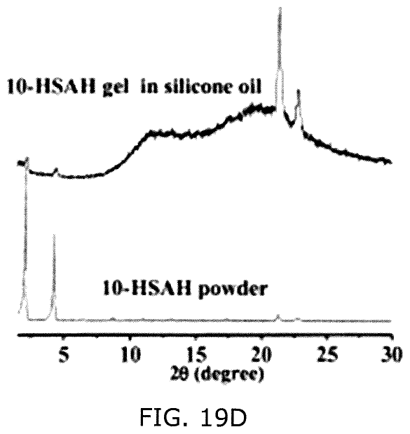

In order to obtain additional insights into the nanostructures of these gels, XRD diffractograms of the organogels and neat solids of n-HSAH (n=0, 2, 6, 10) were also compared (FIGS. 19A-19D and Table 3). The d-values for n-HSAH (n=0, 2, 6, 10) powders from the Bragg relationship were in specific ratios, such as 1:1/2:1/3:1/4 or 1:1/2:1/4:1/5, indicating lamellar stacking. Although the d-spacing (53.83 Å) for neat 0-HSAH was about twice of the calculated extended molecular length (27.62×2 Å), indicating bilayer packing, the d-spacings of the 2-HSAH (30.96 Å), 6-HSAH (35.63 Å) and 10-HSAH (41.76 Å) were near the length of one extended molecule (30.21 Å, 35.23 Å, and 40.23 Å, respectively), indicating single molecule lamellar stacking.

Attempts to index the powder diffraction peaks of 0-HSAH in order to assign a general cell packing were unsuccessful due to the presence of only 8 peaks. However, it was possible to make a preliminary assignment from analyses of FTIR data. For 2-HSAH (13 peaks), orthorhombic packing (a=8.64 Å, b=17.77 Å, c=30.94 Å); For 6-HSAH (14 peaks), orthorhombic packing (a=8.42 Å, b=24.11 Å, c=35.12 Å); For 10-HSAH (16 peaks), orthorhombic packing (a=15.53 Å, b=6.87 Å, c=41.57 Å); The low angle peaks in the XRD diffractograms of 10 wt % n-HSAH (n=0, 2, 6, 10) in silicone oil gels were very close to those of the neat powders. This similarity strongly suggests that the organizations of molecules in the SAFINs of the silicone oil gels and neat powders are very similar.

TABLE 3
XRD data and analyses.

| | | | | | Probable cell |
|---|---|---|---|---|---|
| | | | | d-value | type and |
| | | d-value | d-value | ratio for | dimensions (Å) |
| | L (Å)^{a} | (Å) for powder | (Å) for gel | powder | for powder |
| 0-HSAH | 27.62 | 53.83, 26.73, 17.25, | 54.28, 17.31, | 1:1/2:1/3:1/4 | Trielinic^{c} |
| | | 12.76, 6.37, 5.06, | 4.36, 3.98 | | |
| | | 4.38, 3.96 | | | |
| 2-HSAH | 30.21 | 30.96, 15.23, 10.08, | 28.68, 14.62, | 1:1/2:1/3:1/4 | Orthorhombic^{b} |
| | | 7.52, 6.98, 6.19, | 7.38, 6.88, | | (a = 8.64, |
| | | 6.01, 5.41, 4.16, | 6.10, 5.91, 4.13, | | b = 17.77, |
| | | 3.91, 3.74, 3.46, 3.37 | 3.88 | | c = 30.94) |
| 6-HSAH | 35.23 | 35.63, 17.51, 8.67, | 33.17, 16.91, | 1:1/2:1/4:1/5 | Orthorhombic^{b} |
| | | 7.76, 7.25, 6.91, | 6.73, 4.17, 3.97 | | (a = 8.42, |
| | | 6.55, 5.75, 5.21, | | | b = 24.11, |
| | | 4.92, 4.17, 3.95, | | | c = 35.12) |
| | | 3.79, 3.45 | | | |
| 10-HSAH | 40.23 | 41.76, 20.46, 13.55, | 40.17, 19.77, | 1:1/2:1/3:1/4 | Orthorhombic^{b} |
| | | 10.11, 8.07, 7.73, | 4.14, 3.89 | | (a = 15.53, |
| | | 7.33, 6.73, 6.19, | | | b = 6.87, |
| | | 5.75, 5.09, 5.03, | | | c = 41.57) |
| | | 4.63, 4.16, 3.90, 3.66 | | | |

^{a}Extended length calculated by MM2 method with Chem 3D Ultra 12.0 software (Cambridge Soft Corporation) and adding the van der Waals radii of the terminal atoms.⁵⁵
^{b}Indexed with JADE 9 software (Materials Data Inc.) .
^{c}From infrared spectroscopic data; vide infra.

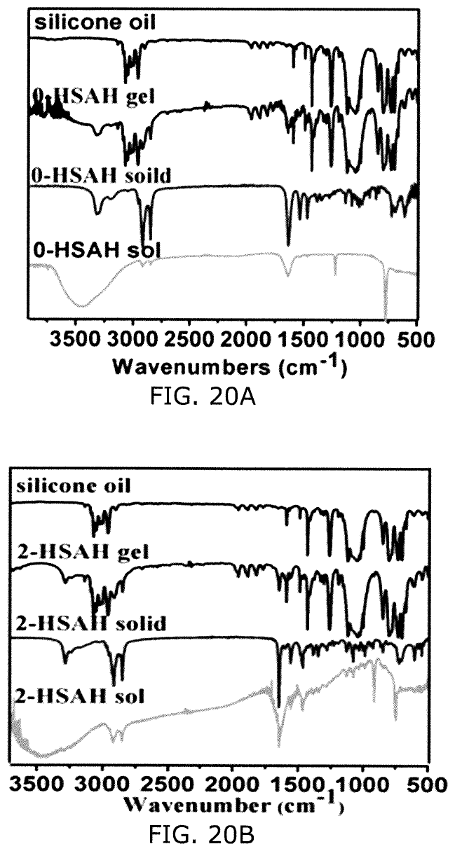

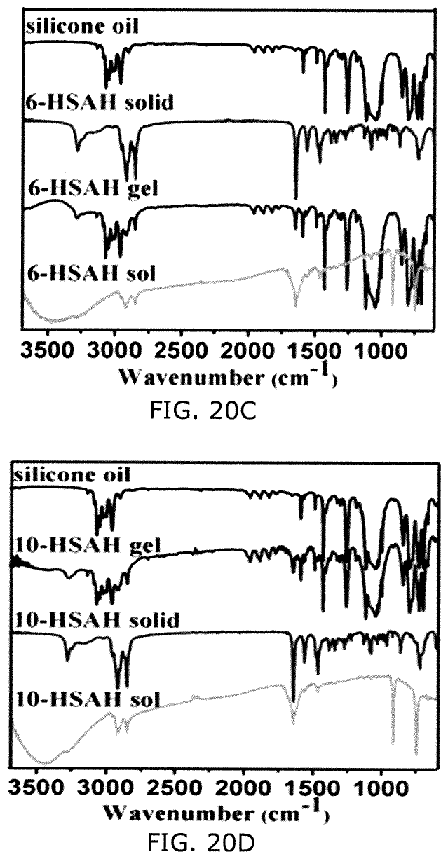

The nearly identical nature of the FT-IR spectra of n-HSAH (n=0, 2, 6, 10) in the silicone oil SAFINs and neat powders (FIGS. 20A-20D) is consistent with the conclusions derived from the XRD data: the SAFINs of the gels and the powders have the same or very similar molecular packing arrangements. For 0-HSAH (FIG. 20A), the OH and NH—NH₂ stretching modes appear in CHCl₃ solution at 3436 cm⁻¹ as a broad band; upon gel formation, they are observed at 3314, 3296 and 3195 cm⁻¹ as separate bands, indicating specific hydrogen-bonding of the OH and NH—NH₂ groups in this SAFIN. Intense bands at 1635 cm⁻¹ and 1535 cm⁻¹ are ascribed to the amide I and amide II vibrations, respectively, and indicate strong hydrogen-bonding interactions between amide groups. The FT-IR spectra of n-HSAHs (n=2, 6, 10) (FIGS. 20B and 20C) are very similar with each other. The broad peaks centered at ˜3440 cm⁻¹ are assigned to the free OH and NH—NH stretching modes of the n-HSAHs (n=2, 6, 10) in their sols. Thus, the peaks at ˜3280, 3238, 1645 and 1558 cm⁻¹ are assigned to hydrogen-bonded OH, NH—NH and CONH stretching modes, respectively. For all the n-HSAH, the CH₂ antisymmetric and symmetric bands appeared at 2917 and 2850 cm⁻¹, respectively, suggesting that the alkyl chains are predominantly in all-trans conformations. However, some differences in the packing between 0-HSAH and n-HSAH (n=2, 6, 10) can be seen in the CH₂ scissoring vibrations near 1470 cm⁻¹ and CH₂ rocking vibrations near 720 cm⁻¹. For Q-HSAH, single peaks were observed at ˜4467 cm⁻¹ and ˜720 cm⁻¹ (indicative of a triclinic matrix), while they split into two peaks at ˜1471/1467 cm⁻¹ and 719/729 cm⁻¹ for n-HSAH (n=2, 6, 10) (as expected for an orthorhombic arrangement).

TABLE 4
Summary of the XRD spectral data according to the Bragg
relationship and calculated extended molecular lengths (L, Å).

| | L (Å) | Characteristic d-values (Å) | Position ratios |
|---|---|---|---|
| 0-HSAH | 27.62 | 53.83, 26.73, 17.25, 12.76 . . . | 1:1/2:1/3:1/4 |
| | | 4.38, 3.96 | |
| (R)-HSA | 26.31 | 49.45, 24.43, 15.83 . . . 4.56, 3.97 | 1:1/2:1/3 |
| 10-HSAH | 40.23 | 41.76, 20.46, 13.55, 10.11 . . . 4.16, | 1:1/2:1/3:1\4 |
| | | 3.90, 3.66 | |
| HSN-5 | 33.6 | 33.47, 16.25, 10.76 . . . 4.16, 3.77 | 1:1/2:1/3 |

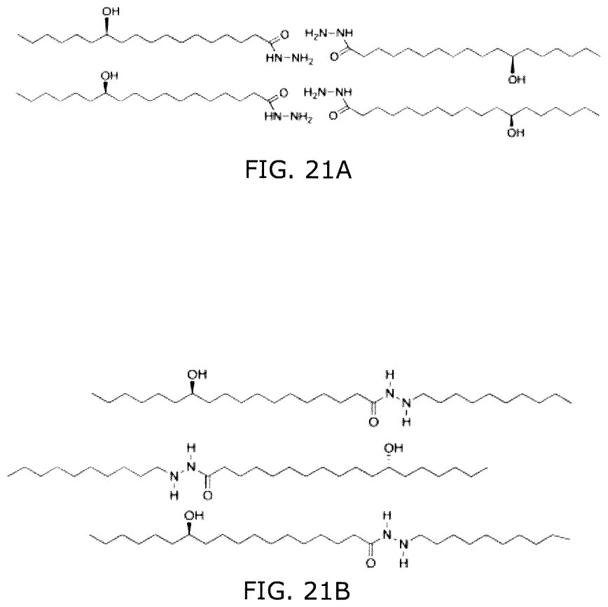

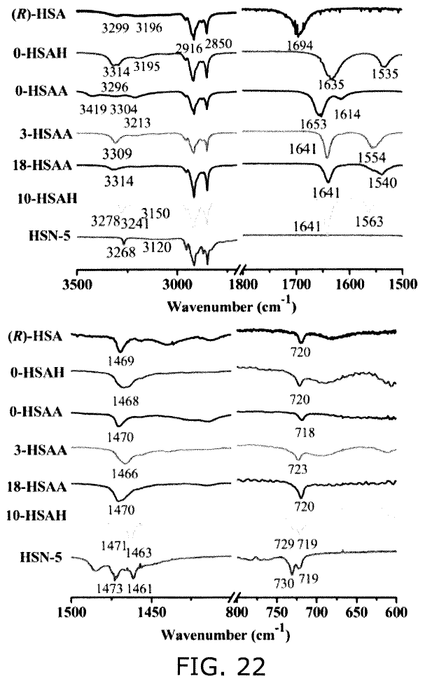

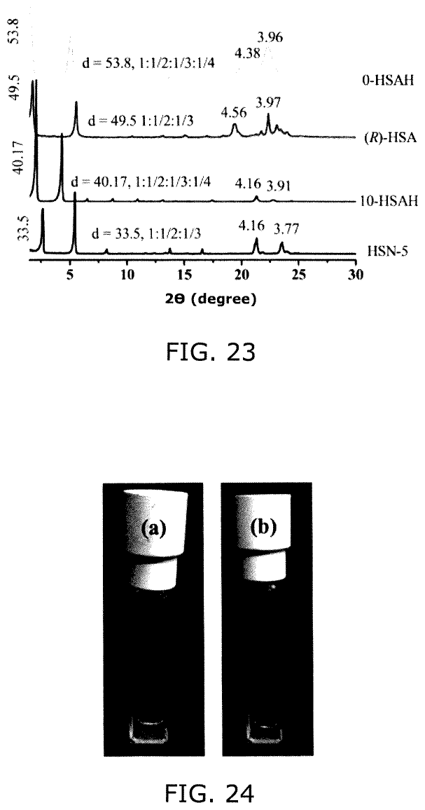

Based on the XRD and FT-IR data, two potential molecular stacking models for 0-HSAH (FIG. 21A) and for n-HSAH (n=2, 6, 10) are proposed. The similarity among the FT-IR band positions of 0-HSAH and (R)-12-hydroxystearamide (0-HSAA) and (R)-HSA (where comparisons can be made) and their XRD d-spacings (FIGS. 22, 23 and Table 4) indicate very similar molecular packing arrangements: for solid (R)-HSA and 0-HSAA, the head groups (—COOH for (R)-HSA and —CONH₂ for 0-HSAA) of the adjacent molecules form strong intermolecular hydrogen-bonding, leading to a bilayered unit as illustrated in the literature. However, the fact that the —OH and —NH stretching vibrations of 0-HSAH are shifted to significantly lower wavenumbers than those of 0-HSAA, indicates stronger hydrogen-bonding interactions among 0-HSAH molecules: the hydrazide groups of 0-HSAH form strong primary hydrogen bonds, leading to a bilayered unit and the pendant hydroxyl groups form weak secondary hydrogen bonds, leading to a 1D hydrogen bonding network (FIG. 21A).

For the other n-HSAH, the packing must be quite different. For example, in 10-HSAH, the frequencies of the —OH and NH—NH bands at 3278, 3241 and 3150 are similar to those of (R)-18-(pentylamino)octadecan-7-ol (HSN-5) (FIG. 22), indicating strong hydrogen-bonding interactions between the —OH and NH—NH groups. The other relevant FT-IR bands in Table 5 and XRD d-spacing (consistent with layers of one molecule in thickness) are quite similar to those of HSN-5 (FIGS. 22 and 23, and Table 4) and (R)-HSA methyl ester. Thus, the packing model proposed for the n-HSAH is like those for HSN-5 and HSA methyl ester: the alkyl chains are again extended in an all-trans conformation and the molecules are in an orthorhombic subcell (FIG. 21B, taking 10-HSAH as an example). The FT-IR and XRD data of the N-alkylated n-HSAH were also compared with those of (R)-12-hydroxy-N-propyloctadecanamide (3-HSAA) and (R)-12-hydroxy-N-octadecyloctadecanamide (18-HSAA) (FIGS. 22 and 23). Although the longest distance d-spacing of 3-HSAA and 18-HSAA are near one extended molecular length, their FT-IR spectra are not similar; they indicate very different packing arrangements from those of the n-HSAH.

TABLE 5
Some important FT-IR peaks from n-HSAH (n = 0, 2, 6, 10) in
their silicone oil gels and neat powders and their assignments.

| Frequency (cm⁻¹) | | | | |
|---|---|---|---|---|
| 0-HSAH | 2-HSAH | 6-HSAH | 10-HSAH | Assignment^{[a]} |
| 3314, 3296, | 3284, 3238, | 3279, 3239 | 3278, 3241 | —OH, NH— |
| 3195 | | | | NH₂/NH—NH |
| 2918 | 2917 | 2917 | 2917 | CH₂, asym str. |
| 2850 | 2850 | 2850 | 2850 | CH₂, sym str. |
| 1635 | 1645 | 1643 | 1641 | Amide I |
| 1535 | 1558 | 1561 | 1563 | Amide II |
| 1467 | 1471, 1463 | 1471, 1463 | 1471, 1463 | CH₂ scissoring |
| 721 | 729, 719 | 729, 719 | 729, 719 | CH₂ rocking |

^{[a]}Extensive peak overlaps preclude unambiguous peak assignments above 3200 cm⁻¹.

### Example 4. Gels as Drug Release Agents

In order to explore the possible application of these gels as drug release agents, the diffusion coefficients for an anionic dye, methylene blue, and a cationic dye, erythrosine B, from 2 wt % 0-HSAH/ethylene glycol gel blocks into ethylene glycol liquids were determined at 25° C. using an early time approximation to Fick's second law (Eq 2). See FIG. 24. The diffusion coefficients of methylene blue and erythrosine B in a 2 wt % 0-HSAH in ethylene glycol gel block were measured spectrophotometrically. The ethylene glycol aliquots covering the gel were replaced at different time intervals and their absorbances were recorded. A total of 9 aliquots was employed for each dye (see Table 6).

$$\frac{{M}_{t}}{{M}_{∞}} = 4 {( \frac{D t}{π {λ}^{2}} )}^{1 / 2} \qquad ( Eq 2 )$$

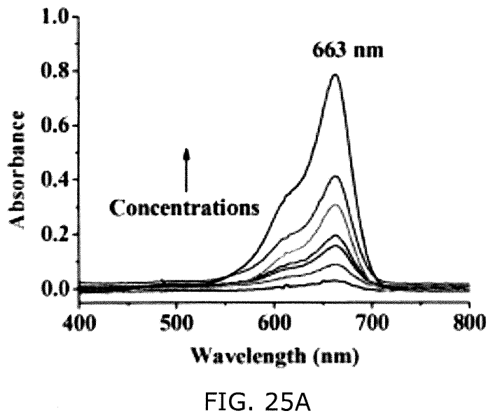

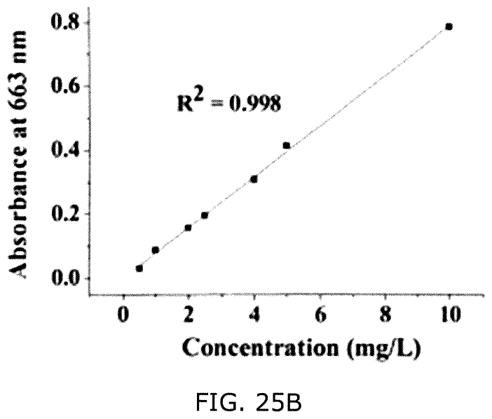

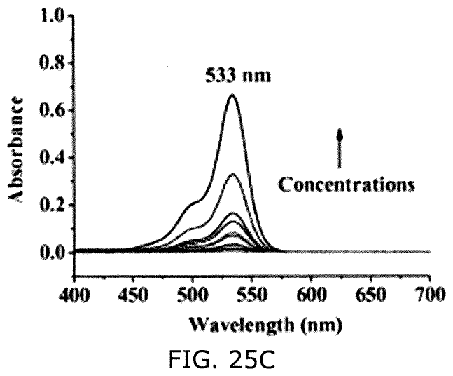

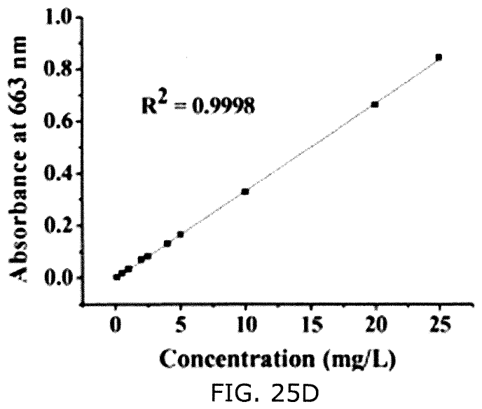

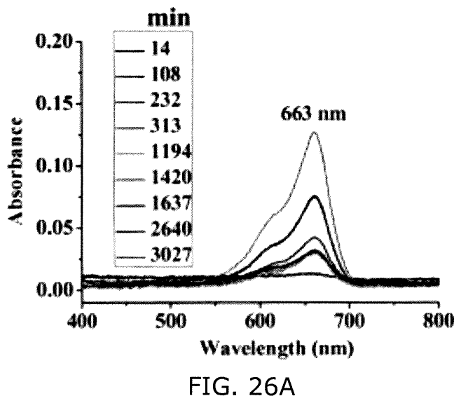

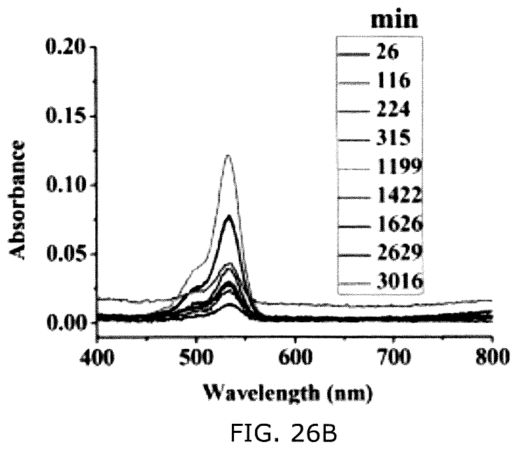

In this equation, Mₜ=the total amount of dye released during the measurement time t (summed for all ethylene glycol aliquots), which can be calculated based on the calibration curve of UV-vis absorbance (FIGS. 25A-25D), the absorbance data obtained in the liquid phases at different times (FIGS. 26A and 26B), and the volume of ethylene glycol liquids (600 μL). M_{∞}=the total amount of dye that was in the gel phase at t=0, Δ=the gel thickness (−0.5 cm), and D=the diffusion coefficient.

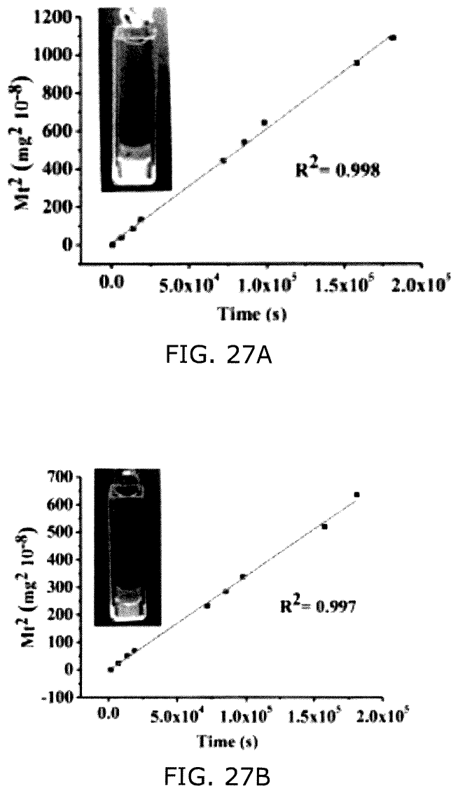

A plot of Mₜ² as a function of t provides a slope, 16DM_{∞}²/Πλ², from which the diffusion coefficient D can be calculated. From the slopes measured in FIGS. 27A and 27B, the diffusion coefficients D of methylene blue and erythrosine B at 25° C. are 7.59×10⁻¹² and 6.04×10⁻¹² m²/s, respectively. Both of these are much smaller than the self-diffusion coefficient of ethylene glycol (9.6×10⁻¹¹ m²/s). After circa 2.5 days, 53% of the anionic and 48% of the cationic dye had been released to the ethylene glycol liquid. These results indicate that many other small charged or uncharged molecules can be slowly released from the gels. For example, gels according to the invention may include a drug dissolved or dispersed therein.

TABLE 6
Contact time and amount of dye diffused
into each aliquot of ethylene glycol.

| | Methylene Blue | | Erythrosine B | |
|---|---|---|---|---|
| | Contact time | Dye content | Contact time | Dye content |
| Aliquot | (min) | (mg × 10⁻⁴) | (min) | (mg × 10⁻⁴) |
| 1 | 14 | 0.8 | 26 | 2.0 |
| 2 | 108 | 5.5 | 116 | 13.6 |
| 3 | 232 | 3.1 | 224 | 6.8 |
| 4 | 313 | 2.2 | 315 | 4.0 |
| 5 | 1194 | 9.5 | 1199 | 21.8 |
| 6 | 1420 | 2.3 | 1422 | 5.2 |
| 7 | 1637 | 2.1 | 1626 | 4.8 |
| 8 | 2640 | 5.6 | 2629 | 14.0 |
| 9 | 3027 | 2.0 | 3016 | 7.6 |

Placing a hydroxyl group along the alkyl chain of stearic acid and transformation of the acid into a hydrazide results in an excellent gelator, (R)-12-hydroxystearic acid hydrazide (0-HSAH) whose gelation properties differ substantially from those of the amide analogue, (R)-12-hydroxystearamide (0-HSAA). 0-HSAH forms gels in ethylene glycol that are self-standing, self-healing, partially thixotropic, moldable and high load-bearing. 0-HSAH also forms self-standing gels in propylene glycol and glycerol. The gelators obtained upon addition of an n-alkyl group to the terminal nitrogen atom of the hydrazide, n-HSAH (n=2, 6, 10), have reduced gelation efficiencies in certain respects, as indicated by the range of liquids gelated and the poorer thermal and mechanical properties of the corresponding gels. However, they are still capable of gelating a variety of liquids at relatively low concentrations, as has been found for the corresponding amides (n-HSAA). Thus, small alterations in the n-HSAH gelator structures cause large changes in the gelation properties.

Without wishing to be bound by any particular explanation, the inventors believe that the presence and length of the N-alkyl groups affect the molecular packing arrangements of the gelator molecules in their SAFINs. Whereas the 0-HSAH molecules are arranged in bilayers with separate H-bonding networks from neighboring hydrazide groups and from the secondary hydroxyl groups along the alkyl chains, the N-alkylated n-HSAH are arranged in orthorhombic subcells with lamellae of one molecular thickness. The subtle structure-property relationships among these (R)-12-hydroxystearic acid hydrazides (as well as between the hydrazides and their amide analogues) indicates how LMWG design can be fine-tuned to produce other new, efficient gelators that form self-standing gels with at least some thixotropic properties.

Notably, the diffusion coefficients of small cationic and anionic dyes in 0-HSAH/ethylene glycol gel blocks are much slower than the self-diffusion coefficient of ethylene glycol. For that reason, gels of 0-HSAH or other n-HSAH compounds are suitable for applications in several areas, including controlled release of drugs incorporated in the gels.

1. A gel composition comprising a liquid solvent and a hydrazide according to formula (I) as shown below: 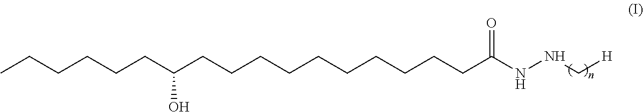
wherein n is an integer from 0 to 20.

2. The gel composition of claim 1, wherein n=0, 2, 6, or 10.

3. The gel composition of claim 1, wherein n=0.

4. The gel composition of any preceding claim, wherein the liquid solvent is chosen from the group consisting of hexane, decane, silicone oil, toluene, CHCl₃, chlorobenzene, ethyl acetate, THF, 1-octanol, 1-butanol, ethanol, methanol, nitrobenzene, DMF, acetonitrile, ethylene glycol, propylene glycol, glycerol, DMSO, propylene carbonate, water, and combinations thereof.

5. The gel composition of claim 3, wherein the liquid solvent is chosen from the group consisting of ethylene glycol, propylene glycol, glycerol, and combinations thereof.

6. The gel composition of claim 4, wherein the liquid solvent is ethylene glycol.

7. The gel composition of claim 4, wherein the liquid solvent is propylene glycol.

8. The gel composition of claim 4, wherein the liquid solvent is glycerol.

9. The gel composition of claim 4, wherein the liquid solvent is silicone oil.

10. A self-standing gel comprising the gel composition according to claim 1.

11. The self-standing gel of claim 10, wherein the gel is substantially thixatropic.

12. The self-standing gel of claim 10, wherein the gel is substantially self-healing.

13. The self-standing gel of claim 10, wherein the gel is substantially moldable.

14. The self-standing gel of claim 10, further comprising charged molecules that can diffuse through the gel.

15. The self-standing gel of claim 10, further comprising a drug dissolved or suspended therein.

16. The gel composition of claim 1, wherein the hydrazide according to formula (I) is the only gelling agent in the composition.
